(19) United States
(12) Patent Application Publication
(10) Pub. No.: US 2022/0081703 A1
(43) Pub. Date: Mar. 17, 2022

(54) METHOD OF QUANTIFYING TARGET SUBSTANCE BY USING GLUCOSE METER AND CELL-FREE PROTEIN SYNTHESIS

(71) Applicant: THE INDUSTRY & ACADEMIC COOPERATION IN CHUNGNAM NATIONAL UNIVESITY (IAC), Daejeon (KR)

(72) Inventors: Dong-Myung KIM, Daejeon (KR); Kyung-Ho LEE, Daejeon (KR); Yeon-Jae JANG, Daejeon (KR)

(21) Appl. No.: 17/420,585

(22) PCT Filed: Jan. 2, 2020
(86) PCT No.: PCT/KR2020/000012

§ 371 Date: Jul. 2, 2021

(30) Foreign Application Priority Data
Jan. 2, 2019 (KR) 10-2019-0000461

(51) Int. Cl. C12Q 1/54

(52) U.S. Cl. CPC C12Q 1/54

(57) ABSTRACT

The present invention relates to a method for quantification of target materials using a glucose meter and cell-free protein reaction in combination. Accordingly, the method for quantification of target materials according to the present invention can quantify target materials with high efficiency in a short time at a low cost, and thus can be useful in various industries.

### TECHNICAL FIELD

The present invention relates to a method for quantification of target materials using a glucose meter and a cell-free protein reaction in combination.

### BACKGROUND ART

A cell-free protein synthesis system was developed to produce only desired proteins without repetitive cell culture steps, and consists of DNA containing target gene sequences, transcription/translation-related protein machinery, nucleic acids, amino acids, energy sources, buffers, etc. The genes encoding the desired proteins are transcribed into mRNA by RNA polymerase, and these mRNAs are translated into proteins by ribosomes, externally supplied tRNA, amino acids, and energy sources, and accordingly, proteins are synthesized through these extracellular transcription and translation processes. Since the cell-free protein synthesis system does not employ living cells, it is advantageous in that various types of proteins can be obtained in a short time.

Meanwhile, the cell-free protein synthesis system has been applied not only to the synthesis of various biomolecules such as antibodies, vaccines, peptides, etc. (Pardee K et al., Portable, On-Demand Biomolecular Manufacturing, Cell. 2016, 167(1):248-259, etc.), but also to tracing of proteins expressed from specific genes (Journal of Biomolecular NMR March 2007, Volume 37, Issue 3, pp. 225-229, etc.). However, research applying the cell-free protein synthesis to quantification of materials is still in its initial stages.

### DISCLOSURE

### Technical Problem

Under the circumstances, the present inventors have made extensive efforts to develop a method for simply quantifying materials, and as a result, they have confirmed that target materials present in an assay sample can be quantitatively and simply analyzed using the characteristics of a cell-free protein synthesis system that determines the progress of a protein synthesis reaction according to the exogenous addition of specific materials in a dependent manner and a glucose meter in combination, thereby completing the present invention.

### Technical Solution

It is one object of the present invention to provide a method for quantification of target materials, including:

(a) performing cell-free protein synthesis by mixing an assay sample containing target materials with a reaction mixture for cell-free protein synthesis;

(b) producing glucose by reacting the synthesized protein with a substrate; and

(c) measuring the level of glucose using a glucose meter.

It is another object of the present invention to provide a method of providing information for diagnosing a disease, including: comparing the concentration of target materials quantified according to the above method with a normal control group.

It is still another object of the present invention to provide a method for screening a material for prevention or treatment of a disease, including: treating a candidate material for prevention or treatment of a disease in an assay sample before Step (a) of the above method.

### Advantageous Effects

The method for quantification of target materials according to the present invention can quantify target materials with high efficiency in a short time at a low cost, and thus can be useful in various industries.

### BRIEF DESCRIPTION OF DRAWINGS

FIG. 1 simply shows the method for quantification of target materials using a reporter protein as an invertase and a glucose meter based on the characteristics of the cell-free protein synthesis reaction that depends on the exogenous addition of amino acids, when the target material is an amino acid.

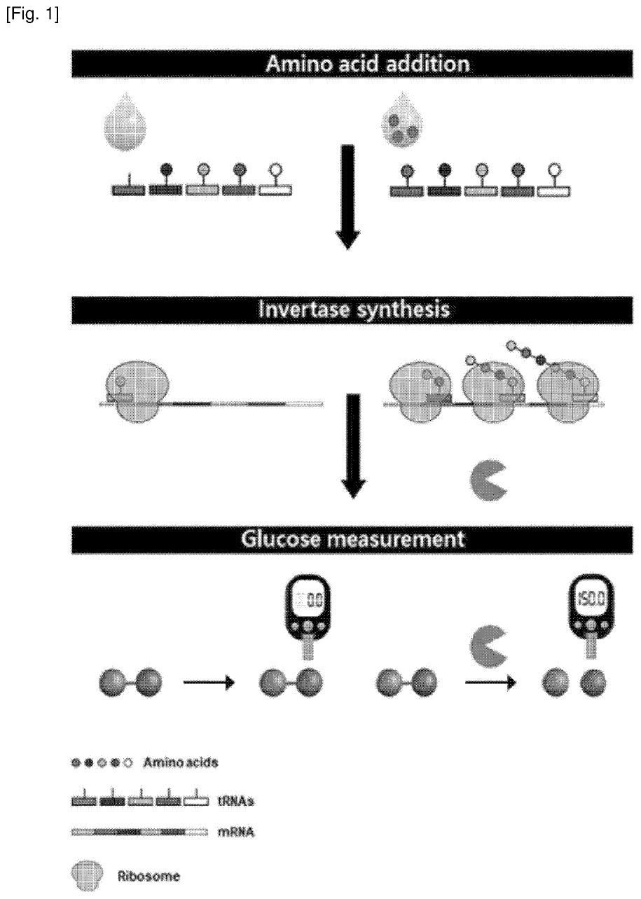

FIG. 2 shows the amount of invertase synthesized through the cell-free protein synthesis reaction. The white bar represents the total amount of invertase, and the black bar represents the amount of soluble invertase.

FIG. 3 shows a readout obtained using a PGM (glucose meter) by reacting invertase synthesized through the cell-free protein synthesis reaction with a 50 mM sucrose solution. It shows the average value of three measurements.

FIG. 4 shows the standard curves of six amino acids relative to the PGM readout.

FIG. 5 confirms the amount of six amino acids contained in heat-treated FBS through CCFPS analysis and PGM readout, while changing the amount of heat-treated FBS. ▴ indicates Ile, Δ indicates Leu, ▪ indicates Lys, □ indicates Tyr, ● indicates Phe, and ∘ indicates Met.

FIG. 6 compares the amino acid concentration measured using PGM (white bar) with the amino acid concentration measured through HPLC standard analysis (black bar).

FIGS. 7A-B confirm that the amount of glucose present in the analyte does not significantly affect the quantification of target materials using the method of the present invention. FIG. 7A shows the added glucose concentration and the amount of invertase, and FIG. 7B shows the result of measuring the amount of glucose according to the incubation time using PGM.

### DETAILED DESCRIPTION OF PREFERRED EMBODIMENTS

Hereinbelow, the present invention will be described in detail. Meanwhile, each of the explanations and exemplary embodiments disclosed herein can be applied to other explanations and exemplary embodiments. That is, all combinations of various factors disclosed herein belong to the scope of the present invention. Furthermore, the scope of the present invention should not be limited by the specific disclosure provided hereinbelow.

Additionally, those of ordinary skill in the art may be able to recognize or confirm, using only conventional experimentation, many equivalents to the particular aspects of the invention described herein. Furthermore, it is also intended that these equivalents be included in the present invention.

One aspect of the present invention provides a method for quantification of target materials, including:

(a) performing cell-free protein synthesis by mixing an assay sample containing target materials with a reaction mixture for cell-free protein synthesis;

(b) producing glucose by reacting the synthesized protein with a substrate; and

(c) measuring the level of glucose using a glucose meter.

In the present invention, there is provided a method for quantification of target materials based on the synthesis reaction of signal-generating proteins, i.e., reporter proteins, using a cell-free protein synthesis reaction.

In the present invention, the term “target material” means a material to be quantified by the method according to the present invention. The “target material” may be a metabolite.

In the present invention, the term “metabolite” refers to a metabolite present in a cell, tissue, organelle, or biological fluid of a living body. The metabolites are substances generated in anabolism and catabolism, and include, without limitation, not only proteins, carbohydrates, lipids, and nucleic acids, which are primary metabolites included in all living cells, but also compounds and secondary metabolites. The metabolites may be, specifically, nucleic acids, compounds, and/or amino acids.

In the present invention, the term “target nucleic acid” means a nucleic acid to be quantified by the method according to the present invention. Specifically, the target nucleic acid may be a sequence present in a virus and/or fusarium, and may be, for example, a Zika virus, an Ebola virus, etc., but is not limited thereto.

In the present invention, the term “target compound” means a compound to be quantified by way of the method according to the present invention. The compound may be, but is not limited to, a compound that binds to a transcriptional and/or translational inhibitor or induces correct folding of a protein.

In the present invention, the term “target amino acid” means an amino acid to be quantified by way of the method according to the present invention. The amino acids are also classified as a type of compound. Specifically, the target amino acid may be at least one selected from the group consisting of arginine, isoleucine, leucine, lysine, methionine, phenylalanine, tyrosine, valine, alanine, cysteine, serine, glycine, histidine, threonine, proline, tryptophan, aspartic acid, asparagine, glutamic acid, and glutamine, and specifically, it may be at least one selected from the group consisting of isoleucine, leucine, lysine, methionine, phenylalanine, and tryptophan, but is not limited thereto.

The cell-free protein synthesis may be carried out depending on the exogenous addition of specific components. For example, the reaction solution of cell-free synthesis excluding a specific component may be mixed with a sample containing the target material to be quantified, which leads to transcription or translation of a protein or correct folding to synthesize a reporter protein, thereby enabling quantification of the target material. The exogenous addition includes both the addition of a sample, which is the target material, to the reaction solution of the cell-free synthesis or the addition of the reaction solution of the cell-free synthesis to the sample, which is the target material.

In one embodiment, when the target material is a nucleic acid, based on the principle of the RNA-toehold switch sensor, a sequence complementary to the nucleic acid to be quantified may be present upstream of the coding region of the reporter protein to form a specific secondary structure in which RNA transcription or translation is inhibited. In this case, when the assay sample containing the nucleic acid to be quantified is mixed with the cell-free reaction solution, protein transcription or translation may occur, resulting in rapid synthesis of the reporter protein. In another embodiment, when the target material is miRNA, a portion of a sequence complementary to the target miRNA may be independently linked downstream of the promoter sequence capable of expressing the reporter protein and the coding region of the reporter protein isolated therefrom. In this case, when the assay sample containing the miRNA to be quantified is mixed with the cell-free reaction solution, the synthesis of the reporter protein may occur by transcription and translation of the protein.

In one embodiment, when the target material is a compound, the compound may be a compound that binds to a transcription inhibitor, a cofactor of a transcription factor, or a compound that otherwise affects the initiation of transcription, or affects the formation of a secondary structure of a protein or correct folding. In this case, the assay sample containing the compound to be quantified may be mixed with the cell-free reaction solution, which leads to protein transcription or translation, or correct folding, resulting in the synthesis of the reporter protein. For example, when the target material is a compound that binds to an aptamer, the principle of reducing the expression level of the reporter protein in the presence of the compound that binds to the aptamer may be employed by inserting the aptamer sequence between the reporter protein coding region and the promoter sequence. In one embodiment, when the target material is an amino acid, the assay sample containing the target amino acid to be quantified may be mixed with the cell-free synthesis reaction solution that does not contain the target amino acid and then incubated, which may result in rapid synthesis of the reporter protein.

In one embodiment, when the metabolite, which is the target material, acts as an antigen for a specific antibody, a capture antibody and a detection antibody that bind to the antigen may be employed following the principle of ELISA, and for example, the reporter protein may be expressed by the reaction mixture of cell-free protein synthesis using a detection antibody conjugated with a DNA sequence encoding the reporter protein.

However, the disclosure described above is merely provided for illustrative purposes, and the present invention is not limited thereto.

The present inventors have developed a method for quantifying target materials, based on the finding that the cell-free protein reaction can be regulated depending on the presence or absence of a specific material.

As used herein, the term “cell-free protein synthesis” refers to performing protein synthesis in vitro, such as in test tubes, etc., which usually takes place within cells, and is the production of a target protein in a short time by extracting only the components essential for protein production, i.e., intracellular protein synthesis apparatus and their factors, from the cell and artificially repeating only the synthesis process of the protein in a state in which the physiological control mechanism of the cell is excluded from the outside of the cell.

In particular, the protein biosynthesis apparatus required for cell-free protein synthesis, i.e., ribosomes, initiation factors, elongation factors, termination factors, aminoacyl tRNA synthetase, RNA polymerase, etc., contained in a cell extract may be used, or may be added separately or produced separately using genetic recombination techniques to be used.

As used herein, the term “reaction mixture for cell-free protein synthesis” refers to a material containing components for performing the cell-free protein synthesis. Specifically, a cell extract or a mixture of recombinant proteins including a protein biosynthesis apparatus such as ribosomes, initiation factors, elongation factors, termination factors, release factors, aminoacyl tRNA synthetase, etc. necessary for protein production may be included, but the reaction mixture for cell-free protein synthesis is not limited thereto, and it may include any component that may be necessary for the synthesis of a reporter protein or detection of a target material.

Specifically, the cell extract may be an extract of wheat germs, rabbit reticulocytes, yeasts, Chinese Hamster ovary cells, or HeLa cells, and more specifically, it may be an E. coli extract, but is no limited thereto as long as it can be used in the cell-free protein synthesis reaction.

Additionally, the recombinant protein may be prepared from the components necessary for protein production by recombinant techniques, and may specifically be an initiation factor such as IF1, IF2, and IF3, an elongation factor such as EF-G, EF-Tu, and EFTs, a release factor such as RF1, RF2, and RF3, a termination factor such as RRF, aminoacyl-tRNA synthetases, methionyl-tRNA transformylase, RNA polymerase, or ribosome. The mixture of recombinant proteins may include all of the components necessary for cell-free protein synthesis, and may specifically include at least one selected from the group consisting of an initiator factor, elongation factor, release factor, termination factor, aminoacyl-tRNA synthetase, methionyl-tRNA transformylase, RNA polymerase, and ribosome.

Meanwhile, when the recombinant protein or a mixture thereof is used, it is possible to exclude enzymes that use the target material as a substrate, and thus the target materials can be quantified.

In one embodiment, the reaction mixture for cell-free protein synthesis may not include the target materials.

For the purpose of the present invention, the reaction mixture for cell-free protein synthesis may include a polynucleotide encoding a reporter protein.

As used herein, the term “reporter protein” is a labelled protein that generates a signal that can easily identify the production or synthesis thereof. In particular, the signal may be in various forms such as luminescence, fluorescence, phosphorescence, color development, electron transfer, etc. In this case, the reporter protein may be a super-folder green fluorescent protein (sfGFP), green fluorescent protein (GFP), yellow fluorescent protein (YFP), red fluorescent protein (RFP), mCherry fluorescent protein, lactamase, galactosidase, horseradish peroxidase (HRP), or glucose oxidase, but is not limited thereto. The fluorescent proteins among the above proteins measure the fluorescence of the fluorescent proteins accumulated in the cell-free synthetic reaction solution, and thus, the activity may be evaluated. Additionally, in the case of the enzymes, the signal can be measured directly by confirming the concentration of the substrate or the product corresponding to each enzyme, but is not limited thereto.

For the purpose of the present invention, the reporter protein may be a glucose-generating enzyme. In this case, a glucose meter, which is widely used worldwide as a personal diagnostic equipment, may be used before the measurement of the signal.

In one example, the glucose-generating enzyme may be an enzyme such as lactase, amylase, sucrase, glucoamylase, glycogen phosphorylase, and/or invertase that dissociates glucose from carbohydrates, disaccharides, and/or polysaccharides containing glucose, such as sucrose, maltose, starch, glycogen, etc., but is not limited thereto. As such, the enzymes that dissociate glucose from carbohydrates, disaccharides, and/or polysaccharides may also be referred to as “glucose dissociation enzymes”. As another example, the glucose-generating enzyme may be an enzyme involved in gluconeogenesis for synthesizing glucose from a substance other than carbohydrates.

Meanwhile, the substrates for the glucose-generating enzymes are known in the art, and after using the enzymes in a reaction for producing glucose from a known substrate and measuring the amount of glucose produced using a glucose meter, the target material in a sample can be quantified using the proportional correlation between (target material)-(synthesized enzyme)-(glucose concentration). For example, when the glucose-generating enzyme is invertase, sucrose, which is the substrate of invertase, may be used as a substrate.

As used herein, the term “polynucleotide encoding a reporter protein” refers to a reporter gene, and may specifically be a DNA (deoxyribonucleic acid) strand of the gene. In particular, the nucleotide sequence of the gene may be obtained from a known database such as GenBank of the NCBI, etc.

In one embodiment, when the target material is a nucleic acid, the polynucleotide encoding the reporter protein may be controlled by an RNA-toehold switch sensor, and a sequence complementary to a target nucleic acid may be present upstream of the polynucleotide encoding the reporter protein, but is not limited thereto.

As used herein, the term “assay sample” refers to a sample containing the target amino acids, which is applied to the method of the present invention.

Specifically, the assay sample may be derived from at least one selected from the group consisting of a feed, food, or chemical substance.

Additionally, the assay sample may be isolated from a living organism, and more specifically, it may be at least one selected from the group consisting of blood, plasma, serum, cancer tissues, and cancer cells isolated from a living organism, or it may be heat-treated and filtered as a pre-treatment, but is not limited thereto.

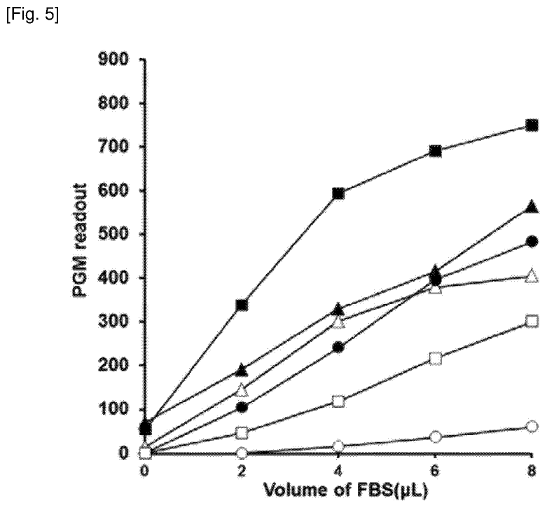

In a specific embodiment of the present invention, the components that interfere with the cell-free protein synthesis reaction were removed by heat-treating FBS, and the resulting aggregates were removed by filtration, and then, the filtered FBS was used in the method of the present invention. As a result, it was confirmed that the amino acids contained in FBS could be quantified (FIG. 5).

For the purpose of the present invention, the assay sample may contain the target materials.

Additionally, the assay sample may be isolated from a patient with a specific disease. The specific disease may be, for example, a disease caused by any one or more of viruses and fungi; an amino acid metabolic disorder; or cancer.

Specifically, the disease caused by the amino acid metabolic disorder may be an amino acid metabolic disorder disease or cancer.

As used herein, the term “amino acid metabolic disorder” is a disease that induces a decrease or an increase in specific amino acids in the body. It is known to occur due to lack or reduced activity of an enzyme involved in a specific amino acid metabolism process, and patients suffering from this disease are clearly distinguished from normal people in terms of the concentration of amino acids in the body. Specifically, the amino acid metabolic disorder may be at least one selected from the group consisting of arginase deficiency, cystinuria, maple syrup urine disease, hyperlysinemia, homocystinuria, hypermethioninemia, and phenylketonuria, but is not limited thereto.

More specifically, the arginase deficiency may be caused by arginine; the cystinuria may be caused by arginine or lysine; the maple syrup urine disease may be caused by isoleucine or valine; the hyperlysinemia may be caused by lysine; the homocystinuria may be caused by methionine; the hypermethioninemia may be caused by methionine; and the phenylketonuria may be caused by phenylalanine or tyrosine, but is not limited thereto.

Additionally, plasma-free amino acids are redistributed or translocated to support the growth and development of cancer, and thus, the concentration (profile) of amino acids in the body of cancer patients is clearly distinguished from those of normal people.

The method for quantification of target materials of the present invention may further include calculating the concentration of the target materials by comparing with the standard concentration curve for each target material. The standard concentration curve may be prepared using a standard sample.

As used herein, the term “standard sample” may include each target material at a specific concentration, and for example, it may contain each target material at a concentration of 0.01 μM to 200 μM, but is not limited thereto.

When the cell-free protein synthesis is performed using the standard sample, the signal of the reporter protein corresponding to the specific concentration of each target material can be determined, and accordingly, the concentration of each target material according to the signal of the protein can be determined. Therefore, the standard concentration curve for each target material according to the signal of the protein includes information on the signal of the protein according to the specific concentration of the target material, and thus the concentration of the target materials can be calculated by comparing the signal intensity measured according to the method for quantification of the present invention with the concentration curve.

Another aspect of the present invention provides a method of providing information for diagnosing a disease, including: comparing the concentration of target materials quantified according to the method for quantification of the present invention with a normal control group. The quantification method, target material, and disease are the same as described above.

For example, when the disease is an amino acid metabolism-related disease, the concentration (profile) of amino acids in the body may be different from those of normal people and patients as a direct or indirect result of the disease, and the specific concentration (profile) of the amino acids exhibited by the disease is known in the art, and accordingly, those skilled in the art can apply the method for quantification of amino acids according to the present invention to diagnose the above diseases or provide information for diagnosis.

Still another aspect of the present invention provides a method for screening a material for prevention or treatment of a disease, including:

(a) treating a candidate material with an assay sample isolated from a living organism;

(b) performing protein synthesis by mixing an assay sample treated with the candidate material and a reaction mixture for cell-free protein synthesis;

(c) producing glucose by reacting the synthesized protein with a substrate and measuring the level of glucose using a glucose meter; and

(d) comparing the level of glucose with a normal control group.

As used herein, the term “candidate material” refers to a material that is expected to be able to treat the above-described disease, and any material that is expected to be able to directly or indirectly alleviate or improve the disease can be used without limitation.

In the present invention, the step (a) of treating a candidate material for the prevention or treatment of a disease with an assay sample may be carried out by way of a method known in the art. As a specific example, the candidate material may be treated by incubating the candidate material with an assay sample, or by administering the material in vivo, but is not limited thereto. Those skilled in the art will be able to employ a method suitable for the purpose of the present invention.

Additionally, the steps (b) and (c) may be carried out by way of the method for quantification of the present invention.

Lastly, the step (d) is a step of deciding whether the candidate material can be used as a prophylactic or therapeutic material for the disease. For example, in the case of the amino acid metabolism-related disease, patients have a concentration (profile) of amino acids in the body which is different from those of normal people, and thus, candidate materials that exhibit the same or similar amino acid concentration as a normal control can be used as a prophylactic or therapeutic material for the amino acid metabolism-related disease.

### MODE FOR CARRYING OUT THE INVENTION

Hereinafter, the present invention will be described in more detail by way of Examples and Experimental Examples. However, these Examples and Experimental Examples are provided for illustrative purposes only, and the scope of the invention is not intended to be limited to or by these Examples and Experimental Examples.

### Example 1: Obtaining Cell Extract for Cell-Free Protein Synthesis

The cell extract S12 used to construct the cell-free protein synthesis system of the present invention was obtained according to the methods disclosed in Korean Patent No. 10-0733712 and Journal of Biotechnology 126 (2006) 554-561. Thereafter, in order to remove the residual amino acids, the S12 extract was centrifuged in a Vivaspin centrifugal concentrator (Sartorius Stedim Biotech GmbH, Gottingen, Germany). After addition of 18 mL of wash buffer (10 mM Tris-acetate, pH 8.2, 14 mM magnesium acetate, 80 mM potassium acetate, and 1 mM dithiothreitol) to 2 mL of the S12 extract, the centrifugation process was repeated 3 times at 2,000 g to reduce the volume of the diluted extract to the original volume (2 mL). Lastly, the thus-prepared S12 extract was frozen using liquid nitrogen and stored at −80° C.

### Example 2: CFPS Reaction: Cell-Free Protein Synthesis Reaction

The cell-free protein synthesis reaction was performed to produce invertase.

First, the reaction mixture of cell-free protein synthesis (hereinafter referred to as “CFPS reaction mixture”) has the following composition: 57 mM HEPES-KOH, pH 8.2; 1.2 mM ATP; 0.85 mM of GTP, UTP and CTP; 80 mM ammonium acetate; 12 mM magnesium acetate; 80 mM potassium acetate; 34 μg/mL 1,5-formyl-5,6,7,8-tetrahydrofolic acid; 2 mM of each amino acid; 2% polyethylene glycol 8000; 3.2 U/mL creatine kinase; 67 mM creatine phosphate; 24% (v/v) diafiltrated S12 extract; and 6.7 μg/mL DNA template.

The DNA of E. coli BL21 strain was amplified by PCR to obtain an ORF of invertase (EC 3.2.1.26), and then cloned into a pK7 vector to prepare a pK7Inv plasmid, which was used as a template for the CFPS reaction.

26 μL of the CFPS reaction mixture without containing the target amino acid, i.e., the reaction mixture of complementary cell-free protein synthesis reaction (CCFPS), was mixed with the assay sample containing 4 μL of the target amino acid. All CFPS and CCFPS reactions were performed in a water bath set at 30° C. for 1 hour.

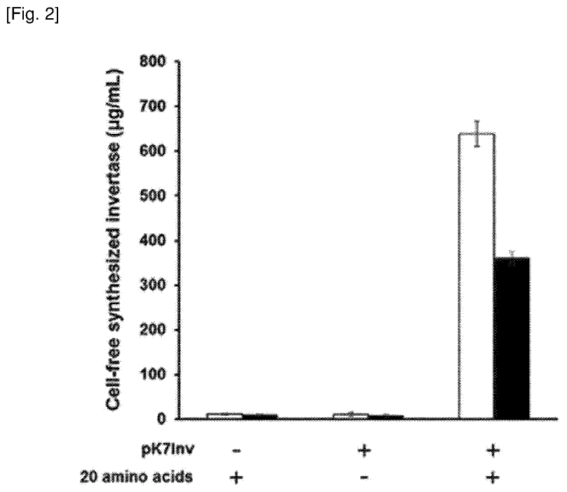

As a result, the amount of invertase synthesis without the addition of external amino acids was similar to that of the reaction without the pK7Inv plasmid. In contrast, the invertase synthesized in the CFPS reaction containing 20 amino acids was about 640 μg/mL and showed a solubility of 56% (FIG. 2).

These results indicate that the invertase synthesis was highly dependent on the presence or absence of amino acids, and that the level of amino acids present in the filtered extract and the activity of invertase were insignificant such that they had no effect on the quantification of amino acids.

### Example 3: Measurement of Amino Acid Levels Using Cell-Free Synthesized Invertase

After completion of the CCFPS reaction in the same manner as in Example 2, 15 μL of the reaction mixture was added to a microtube containing 15 μL of 100 mM sucrose solution in 50 mM Tris-acetate buffer (pH 6.0). After incubation at 37° C. for 10 minutes, the reaction mixture was heated at 90° C. for 10 minutes to terminate the enzymatic hydrolysis reaction, that is, a reaction in which glucose was produced by hydrolyzing sucrose.

After centrifugation at 3,000 g, 5 μL of the supernatant was harvested and loaded onto a glucose meter (Accu-Check Inform II, Roche Diagnostics, Mannheim, Germany) strip to measure the resulting glucose level. The concentration of amino acids in the sample was analyzed by comparing the measured values of the glucose meter with the standard curve.

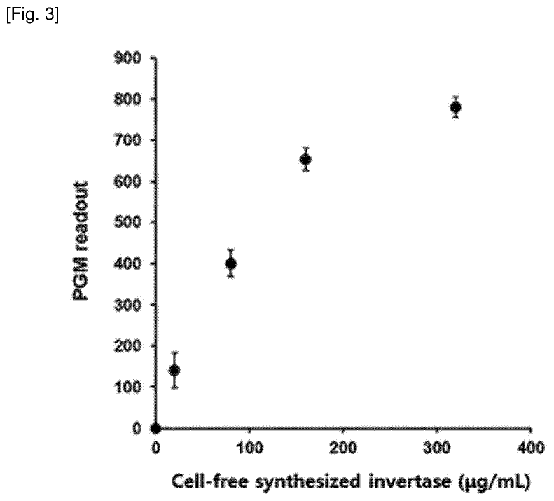

As a result, it was confirmed that the PGM readouts increased in proportion to the concentration of the cell-free synthesized invertase up to a level of about 200 μg/mL (FIG. 3). In combination with the results of Example 2, it can be found that PGM can be used for the quantification of amino acids.

### Example 4: Measurement of Level of Six Amino Acids Using Cell-Free Synthetized Invertase

Based on the above experimental results, PGM was applied to the quantification of amino acids contained in FBS using six amino acids (isoleucine, leucine, lysine, methionine, phenylalanine, and tyrosine) that are associated with various metabolic disorders as target amino acids.

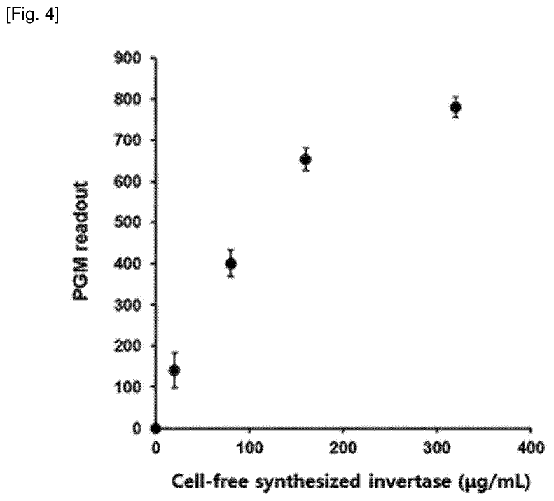

First, in the same manner as in Example 3, a standard curve of six amino acids was prepared relative to the PGM readouts. Each amino acid showed a result that was directly proportional to the PGM readouts up to a concentration of 20 μM, and the minimum detection level (limit of detection) was 0.1 μM to 1.5 μM (FIG. 4).

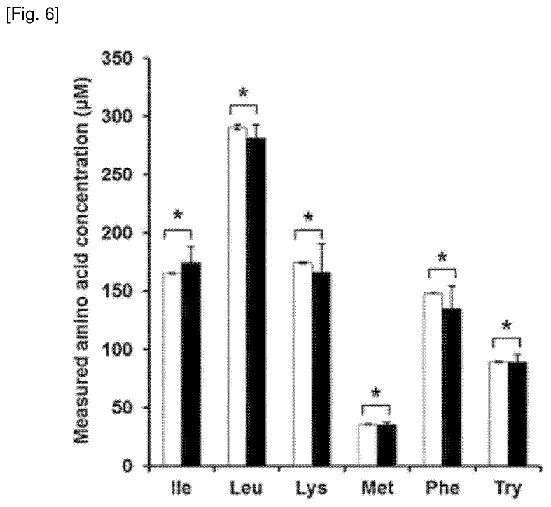

Next, for the analysis of amino acids in FBS, the amino acids were heated at 90° C. for 10 minutes to inactivate the enzymes interfering with protein synthesis and filtered to remove aggregated proteins, and thereafter, the filtrate was added to the CCFPS analysis reaction mixture (i.e., CFPS reaction mixture not containing the amino acids to be analyzed) and analyzed using a Hitachi L-8900 amino acid analyzer (Hitachi High-Technologies, Tokyo, Japan). As a result, it was confirmed that the PGM readouts increased in proportion to the amount of FBS (FIG. 5). Additionally, it was confirmed that the result of measuring the amino acid concentration did not show a significant difference even compared with the HPLC analysis result (FIG. 6).

Meanwhile, considering that most biological samples contain glucose, an experiment was performed using samples containing various concentrations of glucose (up to 10 mM (150 mg/dL)).

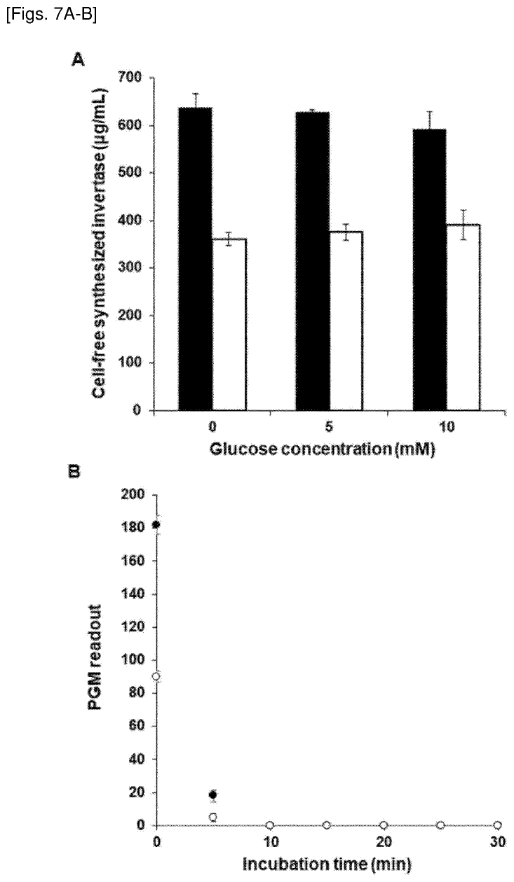

As a result, it was confirmed that glucose was degraded during the incubation process for the cell-free protein synthesis, and accordingly, it was found that even when the biological samples contained glucose, they had no effect on the PGM measurement value (FIG. 7).

Through the experimental results, it was confirmed that the metabolites can be quantified using the cell-free synthesis of invertase, a representative glucose-generating enzyme. Similarly, with respect to other metabolites, nucleic acids and compounds, the amount of the target materials contained in the assay sample can be quantified by way of the cell-free protein synthesis as in the above-described embodiment according to the contents disclosed herein.

Those of ordinary skill in the art will recognize that the present invention may be embodied in other specific forms without departing from its spirit or essential characteristics. The described embodiments are to be considered in all respects only as illustrative and not restrictive. The scope of the present invention is therefore indicated by the appended claims rather than by the foregoing description. All changes which come within the meaning and range of equivalency of the claims are to be embraced within the scope of the present invention.

1. A method for quantification of target materials, comprising:
(a) performing cell-free protein synthesis by mixing an assay sample containing target materials with a reaction mixture for cell-free protein synthesis;
(b) producing glucose by reacting the synthesized protein with a substrate; and
(c) measuring the level of glucose using a glucose meter.

2. The method of claim 1, wherein the reaction mixture for the cell-free protein synthesis of Step (a) comprises a polynucleotide encoding glucose-producing enzymes.

3. The method of claim 1, wherein the reaction mixture for the cell-free protein synthesis of Step (a) comprises a polynucleotide encoding any one or more enzymes selected from lactase, amylase, sucrase, glucoamylase, and invertase.

4. The method of claim 1, wherein the synthesized protein of Step (b) is selected from lactase, amylase, sucrase, glucoamylase, and invertase.

5. The method of claim 1, wherein the target material is selected from amino acids, compounds, and nucleic acids.

6. The method of claim 1, comprising calculating the concentration of target materials by comparing with the standard concentration curve for each target material.

7. The method of claim 1, wherein the reaction mixture for the cell-free protein synthesis comprises a cell extract or a mixture of recombinant proteins.

8. The method of claim 7, wherein (i) the cell extract is any one selected from an extract of E. coli, wheat germs, rabbit reticulocytes, yeasts, Chinese Hamster ovary cells, or HeLa cells; and
(ii) the recombinant protein is at least one selected from the group consisting of an initiation factor, elongation factor, release factor, termination factor, aminoacyl-tRNA synthetase, methionyl-tRNA transformylase, RNA polymerase, and ribosome.

9. The method of claim 1, wherein the assay sample is derived from at least one selected from a feed, food, and chemical substance.

10. The method of claim 1, wherein the assay sample is isolated from a living organism.

11. The method of claim 10, wherein the assay sample is heat-treated and filtered.

12. A method of providing information for diagnosing a disease, comprising: comparing the concentration of target materials quantified according to the method of claim 10 with a normal control group.

13. The method of claim 12, wherein the disease is a disease caused by any one or more of viruses and fungi; an amino acid metabolic disorder; or cancer.

14. A method for screening a material for prevention or treatment of a disease, comprising:
(a) treating a candidate material with an assay sample isolated from a living organism;
(b) performing protein synthesis by mixing an assay sample treated with the candidate material and a reaction mixture for cell-free protein synthesis;
(c) producing glucose by reacting the synthesized protein with a substrate and measuring the level of glucose using a glucose meter; and
(d) comparing the level of glucose with a normal control group.

15. The method of claim 14, wherein the disease is a disease caused by any one or more of viruses and fungi; an amino acid metabolic disorder; or cancer.
